(19) United States
(12) Patent Application Publication
(10) Pub. No.: US 2023/0175080 A1
(43) Pub. Date: Jun. 8, 2023

(54) SAMPLING KIT USED IN DETERMINATION OF RESPIRATORY INFECTION

(71) Applicant: SEEGENE, INC., Seoul (KR)

(72) Inventor: Jong Yoon CHUN, Seoul (KR)

(73) Assignee: SEEGENE, INC., Seoul (KR)

(21) Appl. No.: 17/911,906

(22) PCT Filed: Mar. 15, 2021
(86) PCT No.: PCT/KR2021/003137

§ 371 Date: Sep. 15, 2022

(30) Foreign Application Priority Data
Mar. 16, 2020 (KR) 10-2020-0032287

(51) Int. Cl. C12Q 1/70; C12Q 1/6844; A61B 10/00

(52) U.S. Cl. CPC C12Q 1/701; C12Q 1/6844; A61B 10/0051; C12Q 1/6806

(57) ABSTRACT

Disclosed is a sampling kit for determining respiratory infection, the kit including: a first vessel containing a washing-out solution; and a second vessel containing a transport medium containing a deactivating agent, so that sampling corresponding to a pre-analytical stage in a protocol for determination of the presence or absence of respiratory infection pathogens can be attained easily, safely, and stably, and self-sampling of a respiratory infection pathogen can be attained.

### BACKGROUND OF THE INVENTION

### 1. Field of the Invention

The present disclosure relates to a sampling kit for determining respiratory infection and a sampling method using same.

### 2. Description of the Prior Art

Respiratory diseases cause serious mortality, especially in children, the elderly, and people with weak immune systems. Identifying causative pathogens is very important for infection control and appropriate patient care. Respiratory pathogen testing has developed rapidly with the development of molecular diagnostic technology, especially real-time PCR. Real-time PCR is more sensitive and prompter and can detect various pathogens simultaneously compared with conventional methods.

Various upper respiratory tract (URT) samples and lower respiratory tract samples have been used for respiratory pathogen testing by molecular diagnosis.

In general, sample types, such as a throat swab and a nasal swab, are widely used as upper respiratory tract samples due to the relatively easy collection and low invasiveness. In addition, a nasopharyngeal aspirate (NPA) as an upper respiratory tract sample can be used in children, but is not viable for adults (Heikkinen et al., 2002; AbuDiab et al., 2008; Chan et al., 2008; Debyle et al., 2012; de la Tabla et al., 2010; Gruteke et al., 2004; Lambert et al., 2008; Meerhoff et al., 2010; Sung et al., 2008).

Meanwhile, sputum and bronchoalveolar lavage (BAL) samples are used as lower respiratory tract samples (Falsey et al., 2012; Branche et al., 2014; Jeong et al., 2014).

In recent years, real-time PCR diagnostic kits are in the limelight due to the 2019-nCoV (new coronavirus) epidemic around the world. For the diagnosis of coronavirus, nasopharyngeal swabs and oropharyngeal swabs are used as upper respiratory tract samples, and sputum and bronchoalveolar lavage are used as lower respiratory tract samples. The nasopharyngeal swab and oropharyngeal swab are obtained by collecting secretions from the nasopharynx and oropharynx using cotton swabs and then stored in universal transport media, and the sputum is collected by rinsing the oral cavity with saline and then stored in a sterile vessel.

However, such upper respiratory tract samples have a risk of infection in a sample collector since the sample collector needs to be in close contact with a patient. In addition, lower respiratory tract samples are difficult to collect and may require pre-treatment before nucleic acid extraction, and especially, sputum samples cannot be applied in the early stages of infection in which patients have substantially no cough symptoms or a dry cough.

As an alternative to these upper and lower respiratory tract samples, a throat wash or a saliva sample has been suggested (Wang et al., Emerging Infectious Diseases, www.cdc.gov/eid, Vol. 10, No. 7, July 2004).

However, the validity for the determination of respiratory infection has not yet been established in a throat wash or a saliva sample, and a sampling kit for effectively collecting, preserving, and transporting a throat wash or a saliva sample has not been disclosed.

Therefore, a novel sampling kit is required that can be used to utilize washing-out samples of the oral cavity and/or throat, rather than upper or lower respiratory tract samples.

Throughout this application, various cited documents and patent documents are referenced and citations are provided in parentheses. The disclosure of cited documents and patents in their entireties are hereby incorporated by reference into this application in order to more fully describe the present disclosure and the art to which the present disclosure pertains.

### SUMMARY OF THE INVENTION

The present inventors endeavored to develop measures whereby in a protocol for determining the presence or absence of respiratory infection pathogens, sampling corresponding to a pre-analytical stage can be easily, safely, and stably attained and, as necessary, self-sampling can be attained. As a result, the present inventors developed a sampling kit including two vessels containing a washing-out solution and a deactivating agent-containing transport medium, respectively. The present inventors verified that the use of a sampling kit of the present disclosure can achieve easy, safe, and stable sampling of respiratory infection pathogens and the use of the samples thus obtained can give real-time amplification signals with excellent sensitivity.

Accordingly, an aspect of the present disclosure is to provide a sampling kit for determining respiratory infection.

Another aspect of the present disclosure is to provide a sampling method for determining respiratory infection.

Still another aspect of the present disclosure is to provide a nucleic acid molecule extraction method for determining respiratory infection.

Another aspect of the present disclosure is to provide a method for determining respiratory infection.

Other purposes and advantages of the present disclosure will be clarified by the following detailed description with the accompanied claims.

In accordance with an aspect of the present disclosure, there is provided a sampling kit for determining respiratory infection, the kit including: (a) a first vessel containing a washing-out solution for washing out a respiratory infection pathogen from the oral cavity and/or throat, the washing-out solution containing an aqueous solution; and

(b) a second vessel containing a transport medium, the transport medium containing a deactivating agent for deactivating the respiratory infection pathogen.

Hereinafter, the present disclosure will be described in detail.

A sampling kit for determining respiratory infection in the present disclosure includes two vessels. A first vessel contains a washing-out solution, and a second vessel contains a transport medium.

The first vessel contains a washing-out solution for washing out a respiratory infection pathogen from the oral cavity and/or throat, the washing-out solution containing an aqueous solution.

The first vessel contains an aqueous solution. An aqueous solution suitable in the present disclosure include any aqueous solution that can wash out a sample from the oral cavity and/or throat.

According to an embodiment of the present disclosure, the aqueous solution of the washing-out solution is a water- or saline-based solution. The saline is more specifically, phosphate buffered saline (PBS) or normal saline, and still more specifically, PBS.

According to an embodiment of the present disclosure, the washing-out solution may further contain at least one component selected from the group consisting of: (a) an alcohol selected from the group consisting of ethanol and isopropanol; and (b) a detergent.

Alcohols are components that are usually contained in typical mouth fresheners, and in the present disclosure, ethanol and isopropanol are suitable, and more specifically ethanol is suitable.

When an alcohol is contained in the washing-out solution, the weight ratio of water and the alcohol is specifically 1:1 to 20:1, and more specifically 3:1 to 10:1. Such a water-alcohol mixture may be contained in a content of 10 to 99 parts by weight relative to 100 parts by weight of the washing-out solution.

The detergent used in the present disclosure may include any detergent that is typically contained in mouth fresheners, and examples thereof may include sodium lauroyl sarcosinate, sodium lauryl sulfate, sodium cocoyl glutamate, sodium myristoyl glutamate, cocamidopropyl betaine, a sucrose fatty acid ester, a sorbitan fatty acid ester, or a poloxamer. In the present disclosure, the detergent can improve sampling efficiency. When contained in the washing-out solution, the detergent may be contained in a content of 0.01 to 10 parts by weight relative to 100 parts by weight of the washing-out solution.

The second vessel contains a transport medium containing a deactivating agent for deactivating a respiratory infection pathogen.

One of the characteristics of the sampling kit of the present disclosure is to contain a transport medium containing a deactivating agent for immediately deactivating a resultant product of washing-out of the oral cavity or throat. The resultant product of washing-out is placed into the transport medium by a user (e.g., a patient oneself or a sample collector) of the sampling kit of the present disclosure. In a conventional method, swab samples or sputum samples are placed into a deactivating agent-containing transport medium. Such a conventional method has problems of separately requiring swab tools, enabling sample collection by only experts, and exposing sample collectors to a considerable risk of infection due to the adoption of aerosol-generating sampling. Another conventional technique discloses that a throat wash and saliva can be used in the detection of SARS coronaviruses (Wei-Kung Wang et al., Emerging Infectious Disease, 10(7):1213(2004)). However, this method employs a manner in which the collected throat wash and saliva are immediately applied to an RNA isolation kit, so that the sample collected from a patient must be immediately applied to the RNA isolation kit, and thus sample collection must be performed at a place where the RNA separation kit is located, or the throat wash and saliva must be transported to a place where the RNA separation kit is located. There is a restriction in sample collection if sample collection must be performed at a place where the RNA separation kit is located, and there is a risk of infection during transport if samples must be transported to a place where the RNA separation kit is located.

The present disclosure provides a measure to obtain a sample for respiratory infection determination easily, safely, and stably and, as necessary, a measure to obtain a sample safely and stably through self-sampling. To this end, the present disclosure provides a feature of including a second vessel containing a transport medium containing a deactivating agent for deactivating a respiratory infection pathogen as well as the first vessel containing the washing-out solution. According to an embodiment, a user performs gargling with the washing-out solution of the first vessel, and the resultant product of washing-out of the oral cavity or throat thus obtained is placed into the second vessel, so that a pathogen-deactivated sample can be obtained easily and safely, and preferably, a nucleic acid material released from a pathogen can be stably maintained, thereby ensuring excellent detection efficiency.

The deactivating agent used in the present disclosure deactivates the following pathogens that infect respiratory tracts: influenza viruses (e.g., influenza A virus and influenza B virus), respiratory syncytial viruses (RSV) (e.g., RSV A and RSV B), adenoviruses, enteroviruses, parainfluenza viruses (PIV) (e.g., PIV 1, PIV 2, PIV 3, and PIV 4), metapneumoviruses (MPV), bocaviruses, rhinoviruses, coronaviruses (e.g., CoV NL63, CoV 229E, CoV OC43, CoV HKU1, SARS-CoV, MERS-CoV, SARS-CoV-2), Mycoplasma pneumoniae, Chlamydophila pneumoniae, Legionella pneumophila, Haemophilus influenzae, Streptococcus pneumoniae, Bordetella pertussis and/or Bordetella parapertussis.

The deactivating agent used in the present disclosure may deactivate viruses or bacteria by cell lysis and/or protein denaturation.

According to an embodiment of the present disclosure, the deactivation of a respiratory infection pathogen by the deactivating agent used in the present disclosure is attained by cell lysis.

According to an embodiment of the present disclosure, the transport medium containing the deactivating agent contains (i) a chaotropic agent. Specifically, the chaotropic agent is guanidine thiocyanate, guanidine isocyanate, or guanidine hydrochloride, and more specifically, guanidine thiocyanate. The chaotropic agent opens microbial cells to induce cell lysis and allow the release of DNA and RNA, and prevents nucleic acid molecules from being degraded by nucleases.

According to an embodiment of the present disclosure, the transport medium further contains at least one component selected from the group consisting of: (ii) a detergent; (iii) a reductant; and (iv) a chelator.

Specifically, the detergent contained in the transport medium is sodium dodecyl sulfate, lithium dodecyl sulfate, sodium taurodeoxycholate, sodium taurocholate, sodium glycocholate, sodium deoxycholate, sodium cholate, sodium alkylbenzene sulfonate, or N-lauroyl sarcosine.

Specifically, the reductant contained in the transport medium is 2-mercaptoethanol, tris(2-carboxyethyl)phosphine, dithiothreitol, or dimethylsulfoxide.

Specifically, the chelator contained in the transport medium is ethylene glycol tetraacetic acid, hydroxyethylethylenediaminetriacetic acid, diethylene triamine pentaacetic acid, N,N-bis(carboxymethyl)glycine, ethylenediaminetetraacetic, citrate anhydrous, sodium citrate, calcium citrate, ammonium citrate, ammonium bicitrate, citric acid, diammonium citrate, ferric ammonium citrate, or lithium citrate.

The transport medium of the present disclosure may contain a buffer component. Examples of a usable buffer may include tris(hydroxymethyl)aminomethane, citrate, 2-(N-morpholino)ethanesulfonic acid, N,N-Bis(2-hydroxyethyl)-2-aminoethanesulfonic acid, 1,3-bis(tris(hydroxymethyl)methyl amino)propane, 4-(2-hydroxyethyl)-1-piperazine ethanesulfonic acid, 3-(N-morpholino)propanesulfonic acid, bicarbonate, and phosphate, but are not limited thereto.

In the transport medium of the present disclosure, the chaotropic agent as a deactivating agent may be contained in an amount of 0.5-6 M, the detergent may be contained in an amount of 0.1-1 wt %, the chelator may be contained in an amount of 0.01-1 mM, the reductant may be contained in an amount of 0.05-0.3 M, and the buffer may be contained in an amount of 10-200 mM.

According to an embodiment of the present disclosure, the transport medium serves as a deactivating function by lysis of the respiratory infection pathogen and a stabilizing function of a nucleic acid material (specifically, DNA or RNA, and more specifically RNA) released from the lysed pathogen.

According to an embodiment of the present disclosure, sampling is attained by applying the washing-out solution of the first vessel to the human oral cavity and/or throat to perform gargling and placing the resultant liquid of gargling into the transport medium of the second vessel.

The kit of the present disclosure can attain sampling by gargling using the washing-out solution of the first vessel.

As used herein, the term “gargling” refers to an act of holding and moving a washing-out solution in the oral cavity and/or throat (more specifically, oral cavity).

The sampling kit of the present disclosure may be applied to the oral cavity or throat. The application of the kit to the throat causes problems of degrading the user's convenience and showing a severe sampling deviation depending on the user. One of the characteristics of the present disclosure is that respiratory infection can be determined with sufficient sensitivity by using only a mouthwash obtained by washing of the oral cavity, and to the best of the knowledge of the present inventors, the suitability of such a mouthwash as a respiratory infection sample has not been known.

More specifically, the kit of the present disclosure is a sampling kit applied to the oral cavity. According to an embodiment of the present disclosure, a mouthwash sample is sampled.

The kit of the present disclosure may easily attain sampling through gargling, and may attain sampling through brushing or gargling and brushing. According to an embodiment of the present disclosure, sampling is attained by applying the washing-out solution of the first vessel to the human oral cavity and/or throat to perform (i) brushing or (ii) gargling and brushing, and placing a resultant liquid of brushing, a resultant liquid of gargling and a resultant liquid of brushing, or a brush in the transport medium of the second vessel. The kit of the present disclosure may further include an oral brush.

The kit of the present disclosure has advantages in that sampling can be attained by a patient oneself as well as an expert. According to an embodiment of the present disclosure, the kit of the present disclosure is a kit for self-sampling. A patient performs gargling with a washing-out solution and places a resultant liquid of gargling into the transport medium of the second vessel, thereby attaining direct sampling.

According to an embodiment of the present disclosure, the kit of the present disclosure is a kit for determining infection with a respiratory virus and/or a respiratory bacterium. For example, the kit of the present disclosure is a kit for determining infection with influenza viruses (e.g., influenza A virus and influenza B virus), respiratory syncytial viruses (RSV) (e.g., RSV A and RSV B), adenoviruses, enteroviruses, parainfluenza viruses (PIV) (e.g., PIV 1, PIV 2, PIV 3, and PIV 4), metapneumoviruses (MPV), bocaviruses, rhinoviruses, coronaviruses (e.g., CoV NL63, CoV 229E, CoV OC43, CoV HKU1, SARS-CoV, MFRS-CoV, SARS-CoV-2), Mycoplasma pneumoniae, Chlamydophila pneumoniae, Legionella pneumophila, Haemophilus influenzae, Streptococcus pneumoniae, Bordetella pertussis and/or Bordetella parapertussis.

According to an embodiment of the present disclosure, the kit of the present disclosure is a kit for determining infection with a respiratory virus including influenza viruses, respiratory syncytial viruses (RSV), adenoviruses, enteroviruses, parainfluenza viruses (PIV), metapneumoviruses (MPV), bocaviruses, rhinoviruses, and/or coronaviruses. More specifically, the kit of the present disclosure is a kit for determining infection with severe acute respiratory syndrome coronavirus 2 (SARS-CoV-2).

According to an embodiment of the present disclosure, the product sampled by the kit of the present disclosure is directly used in nucleic acid separation without additional cell lysis. According to a general infection determination process, the collected sample is subjected to cell lysis and then nucleic acid separation. When the transport medium of the kit of the present disclosure contains a cell lysis agent as a deactivating agent, the sampled product can be directly used in nucleic acid separation without additional cell lysis. For example, as for nucleic acid separation using magnetic particles, the sampled product is applied to a binding buffer to allow the nucleic acid molecules in the sampled product to bind to the magnetic particles, followed by eluting, thereby obtaining nucleic acid molecules.

According to an embodiment of the present disclosure, nucleic acid separation is performed using magnetic particles, and the sampled product is applied to a binding buffer containing magnetic particles.

According to an embodiment of the present disclosure, the kit of the present is applied to non-aerosol generating sampling.

The term “aerosol generating sampling” refers to a sampling method that is generally applied to a conventional respiratory infection sampling, wherein aerosol generation is promoted by cough stimulation, and includes for example nasopharyngeal swabbing and throat swabbing.

The kit of the present disclosure can attain sampling by applying the washing-out solution of the first vessel to the human oral cavity and/or throat to perform gargling and placing a resultant liquid of gargling into the transport medium of the second vessel, and thus the kit of the present disclosure can be used completely freely from aerosols from patients.

The kit of the present disclosure is suitable to provide a sample used in molecular diagnosis involving nucleic acid amplification.

According to an embodiment of the present disclosure, the real-time nucleic acid amplification reaction to which a sample generated by the kit is applied shows a sensitivity of 10-100 copies/reaction. Specifically, the sensitivity described herein is based on a reaction solution of 20-30 μL for real-time nucleic acid amplification.

In accordance with another aspect of the present disclosure, there is provided a sampling method for determining respiratory infection, the method including: (a) placing a resultant liquid of gargling, which is obtained by applying a washing-out solution containing an aqueous solution to the oral cavity and/or throat and performing gargling, into a transport medium containing a deactivating agent for deactivating a respiratory infection pathogen.

In accordance with still another aspect of the present disclosure, there is provided a nucleic acid molecule extraction method for determining respiratory infection, the method including:

(a) applying a resultant product of the sampling method to a binding buffer containing magnetic particles and binding nucleic acid molecules in the resultant product to the magnetic particles; and

(b) subjecting the nucleic acid molecules bound to the magnetic particles to elution, thereby extracting the nucleic acid molecules for respiratory infection determination.

In accordance with still another aspect of the present disclosure, there is provided a method for determining respiratory infection, the method including:

(a) applying nucleic acid molecules, extracted by the present inventive method, to a real-time amplification solution; and

(b) detecting a signal, generated by performing real-time amplification using the real-time amplification solution, to determine the presence or absence of respiratory infection.

Since the method of the present disclosure uses the sampling kit, descriptions of overlapping contents therebetween will be omitted to avoid excessive complexity of the specification.

The sampling method of the present disclosure further includes a step of resting the transport medium, into which the resultant liquid of gargling has been placed, for a time sufficient to kill and lyse a pathogen and to denature or deactivate proteins, enzymes and/or nucleases of the pathogen. For example, the above-described deactivation is attained by resting the transport medium, into which the resultant liquid of gargling is placed, at room temperature for 0.5-24 hours.

According to an embodiment of the present disclosure, the respiratory infection determination method shows a sensitivity of 10-100 copies/reaction.

According to an embodiment of the present invention, the method of the present invention is a method for determining infection with SARS-CoV-2.

Features and advantages of the present invention are summarized as follows.

(a) The present disclosure presents a measure to easily, safely, and stably attain sampling corresponding to a pre-analytical stage in a protocol for determination of the presence or absence of respiratory infection pathogens.

(b) The feature of the present disclosure is that sampling is attained by using an oral cavity and/or throat wash obtained by applying the washing-out solution and placing the wash into a deactivating agent-containing transport medium. These technical features ensure easiness, safety, and stability of the above-described sampling.

(c) The present disclosure enables self-sampling of a respiratory infection pathogen.

(d) In cases where the kit of the present disclosure is used, sampling is attained by allowing a user to perform gargling with the washing-out solution of the first vessel without a swabbing tool and placing the resultant product of washing-out of the oral cavity or throat into the second vessel, and thus sampling can be attained very easily.

(e) According to the present disclosure, sampling is attained by a simple gargling action and adopts a non-aerosol generating manner, leading to improved safety, and the resultant product of washing-out is placed into the second vessel, and then is immediately biologically deactivated and then transported, or is biologically deactivated during transport, thereby significantly improving safety.

(g) The nucleic acid molecule sample obtained from the sampling kit of the present disclosure can maintain integrity without degradation, so that respiratory infection pathogens can be detected with excellent sensitivity through real-time amplification.

### DETAILED DESCRIPTION OF THE EXEMPLARY EMBODIMENTS

Hereinafter, the present disclosure will be described in detail through examples. It would be obvious to those skilled in the art that these examples are intended to be more concretely illustrative and the scope of the present invention as set forth in the appended claims is not limited to or by the examples.

### EXAMPLES

### Example 1

### Respiratory Infection Pathogen Detection 1 Using Washing-Out Sample Collection Manner

The present inventors collected samples from the respiratory organ in a washing-out manner (i.e., gargling manner) but not a swab manner, and investigated whether the presence or absence of a respiratory infection pathogen was detectable.
(1) Collection of Samples

Samples were collected in a washing-out manner from ten patients infected with influenza A virus. Each patient gargled with 4 mL of saline so as to collect samples in the oral cavity and throat. As for a control, samples were collected from ten non-infected subjects in the same manner above.
(2) Deactivation of Gargle Samples

Each gargle sample was spat into an empty vessel, and the gargle sample was mixed with 2 mL of a transport medium solution containing a lysis component (3 M guanidine thiocyanate, 0.5 wt % sodium dodecyl sulfate, and 120 mM Tris buffer, pH 7.2).
(3) Extraction of Nucleic Acid

The extraction of nucleic acid was carried out using the STARMag 96X4 Universal Cartridge Kit (Cat. No. 744300.4.UC384, Seegene Inc.) and the automatic nucleic acid extraction system Microlab NIMBUS (Cat. No. 65415-02, Hamilton). The extraction was carried out according to the manufacturer of an extraction reagent and the operation manual of the system.

300 μL of the deactivated sample contained in the transport medium was placed into the automatic nucleic acid extraction system.

The extraction reagent contains a lysis buffer, proteinase K, a binding buffer, washing buffer 1, washing buffer 2, washing buffer 3, a elution solution, and magnetic beads.
(4) Detection of Nucleic Acid

The presence or absence of the pathogen was tested using the resultant product of nucleic acid extraction.

A reaction mixture for detection was prepared using Allplex™ respiratory panel 1 Assay reagent (Cat. No. RP9801X, Seegene, Inc.) and the automatic nucleic acid extraction system Microlab NIMBUS (Cat. No. 65415-02, Hamilton). The extraction was carried out according to the manufacturer of an extraction reagent and the operation manual of the system. An internal control (IC) provided from the reagent manufacturer was used according to a protocol.

The reaction mixture was prepared by mixing 5 μL of 5×RP1 MOM, 5 μL of RNase-free Water, 5 μL of 5×Real-time One-Step Buffer, 2 μL of Real-time One-Step Enzyme, and 8 μL of a nucleic acid extract.

PCR plates containing the reaction mixture were subjected to real-time PCR using a real-time PCR machine (CFX96 Real-time cycler, Bio-Rad, US).

The amplification was conducted by reaction at 50° C. for 20 min, reaction 95° C. for 15 min, and 45 cycles of reaction at 95° C. for 10 sec, at 60° C. for 60 sec, and at 72° C. for 10 sec. The fluorescence was measured at 60° C. and 72° C. every cycle.

Data were analyzed by Seegene Viewer S/W (Seegene, Korea).

As a result of analysis, nine samples out of ten patient samples showed amplification results exceeding the threshold at a Ct value between 25 and 32, each indicating a positive result on the respiratory pathogen. Ten out of ten non-infected subjects showed negative results. That is, the washing-out sampling manner can exhibit a clinical sensitivity of 90% and a clinical specificity of 100%, and is very effective in the detection of respiratory pathogens.

### Example 2

### Respiratory Infection Pathogen Detection 2 Using Washing-Out Sample Collection Manner

The present inventors investigated whether the presence or absence of a respiratory infection pathogen was detectable by using an extraction manner without separate lysis in the nucleic acid extraction procedure while a pathogen was deactivated using a transport medium solution containing a lysis component.

The same samples, reagents, and systems as in Example 1 were used except for the following changes in the extraction procedure. That is, the extraction procedure of the extraction system in Example 1 was changed such that, instead of mixing with the lysis buffer, a binding buffer having the same volume as a lysis buffer to be used was additionally mixed.

The preparation and detection of the reaction mixture for detection were also carried out in the same manner as in Example 1.

As a result of testing, nine samples out of ten patient samples showed amplification results exceeding the threshold at a Ct value between 30 and 38, each indicating a positive result on the respiratory pathogen. Ten samples out of ten non-infected subjects showed negative results. That is, when samples were collected in the washing-out sampling manner of the present disclosure and the deactivating transport solution contains a lysis component, such a method exhibited a clinical sensitivity of 90% and a clinical specificity of 100%, even without additional lysis in the extraction procedure, and is very effective in the detection of respiratory pathogens.

### Comparative Example 1

### Respiratory Infection Pathogen Detection Using Swab Sample Collection Manner

As for a control, samples were collected from respiratory organs in a swab manner.
(1) Collection of Samples

Samples were collected by inserting a swap into the nose of the same patient as in Example 1 above.
(2) Preservation of Swab Samples

Each swab was stored in 3 mL of PBS solution.
(3) Extraction and Detection of Nucleic Acid

Extraction and detection were conducted using the same reagents and systems as in the method described in Example 1 above. 300 μL of the samples contained in the transport medium were placed into the automatic nucleic acid extraction system.

As a result of analysis, ten out of ten patient samples showed amplification results exceeding the threshold at a Ct value between 22 and 30, each indicating a positive result on respiratory pathogens. Ten samples out of ten non-infected subjects showed negative results. That is, the conventional swab sampling manner exhibited a clinical sensitivity of 100% and a clinical specificity of 100%.

What is claimed is:

1. A sampling kit for determining respiratory infection, the kit comprising:
(a) a first vessel containing a washing-out solution for washing out a respiratory infection pathogen from the oral cavity and/or throat, the washing-out solution containing an aqueous solution; and
(b) a second vessel containing a transport medium, the transport medium containing a deactivating agent for deactivating the respiratory infection pathogen.

2. The kit of claim 1, wherein the aqueous solution of the washing-out solution is a water- or saline-based solution.

3. The kit of claim 2, wherein the saline is phosphate buffered saline (PBS) or normal saline.

4. The kit of claim 3, wherein the saline is PBS.

5. The kit of claim 1, wherein the washing-out solution further contains at least one component selected from the group consisting of: (a) an alcohol selected from the group consisting of ethanol and isopropanol; and (b) a detergent.

6. The kit of claim 1, wherein the transport medium containing the deactivating agent comprises (i) a chaotropic agent.

7. The kit of claim 6, wherein the transport medium further contains at least one component selected from the group consisting of: (ii) a detergent; (iii) a reductant; and (iv) a chelator.

8. The kit of claim 6, wherein the transport medium serves as a deactivating function by lysis of the respiratory infection pathogen and a stabilizing function of a nucleic acid material released from the lysed pathogen.

9. The kit of claim 6, wherein the chaotropic agent is guanidine thiocyanate, guanidine isocyanate, or guanidine hydrochloride.

10. The kit of claim 7, wherein the detergent is sodium dodecyl sulfate, lithium dodecyl sulfate, sodium taurodeoxycholate, sodium taurocholate, sodium glycocholate, sodium deoxycholate, sodium cholate, sodium alkylbenzene sulfonate, or N-lauroyl sarcosine.

11. The kit of claim 7, wherein the reductant is 2-mercaptoethanol, tris(2-carboxyethyl)phosphine, dithiothreitol, or dimethylsulfoxide.

12. The kit of claim 7, wherein the chelator is ethylene glycol tetraacetic acid, hydroxyethylethylenediaminetriacetic acid, diethylene triamine pentaacetic acid, N,N-bis(carboxymethyl)glycine, ethylenediaminetetraacetic, citrate anhydrous, sodium citrate, calcium citrate, ammonium citrate, ammonium bicitrate, citric acid, diammonium citrate, ferric ammonium citrate, or lithium citrate.

13. The kit of claim 1, wherein sampling is attained by applying the washing-out solution of the first vessel to the human oral cavity and/or throat to perform gargling and placing a resultant liquid of gargling into the transport medium of the second vessel.

14. The kit of claim 13, wherein the kit is configured to sample a mouthwash sample.

15. The kit of claim 1, wherein sampling is attained by applying the washing-out solution of the first vessel to the human oral cavity and/or throat to perform (i) brushing or (ii) gargling and brushing, and placing a resultant liquid of brushing, a resultant liquid of gargling and a resultant liquid of brushing, or a brush into the transport medium of the second vessel.

16. The kit of claim 15, wherein the kit further comprises an oral brush.

17. The kit of claim 1, wherein the kit is a kit for self-sampling.

18. The kit of claim 1, wherein the kit is for determining infection with a respiratory virus and/or a respiratory bacterium.

19. The kit of claim 18, wherein the respiratory virus is an influenza virus, a respiratory syncytial virus (RSV), an adenovirus, an enterovirus, a parainfluenza virus (PIV), a metapneumovirus (MPV), a bocavirus, a rhinovirus, and/or a coronavirus.

20. The kit of claim 19, wherein the coronavirus is severe acute respiratory syndrome coronavirus 2 (SARS-CoV-2).

21. The kit of claim 1, wherein the product sampled by the kit is directly used in the separation of a nucleic acid without additional cell lysis.

22. The kit of claim 21, wherein the nucleic acid is separated using magnetic particles and the sampled product is applied to a binding buffer containing magnetic particles.

23. The kit of claim 1, wherein the kit is applied to non-aerosol generating sampling.

24. The kit of claim 1, wherein the kit is for obtaining a sample used for real-time nucleic acid amplification involving nucleic acid amplification.

25. A sampling method for determining respiratory infection, the method comprising:
(a) placing a resultant liquid of gargling, which is obtained by applying a washing-out solution containing an aqueous solution to the oral cavity and/or throat and performing gargling, into a transport medium containing a deactivating agent for deactivating a respiratory infection pathogen.

26. A nucleic acid molecule extraction method for determining respiratory infection, the method comprising:
(a) applying a resultant product of the method of claim 25 to a binding buffer containing magnetic particles and binding nucleic acid molecules in the resultant product to the magnetic particles; and
(b) subjecting the nucleic acid molecules bound to the magnetic particles to elution, thereby extracting the nucleic acid molecules for respiratory infection determination.

27. A method for determining respiratory infection, the method comprising:
(a) applying nucleic acid molecules, extracted by the method of claim 26, to a real-time amplification solution; and
(b) detecting a signal, generated by performing real-time amplification using the real-time amplification solution, to determine the presence or absence of respiratory infection.
